(19) United States
(12) Patent Application Publication
(10) Pub. No.: US 2019/0110853 A1
(43) Pub. Date: Apr. 18, 2019

(54) A CONCERTINAED EXPANDABLE TISSUE EXPANDER

(71) Applicant: OXTEX LIMITED, Oxfordshire (GB)

(72) Inventor: David Edward JACKSON, Oxfordshire (GB)

(21) Appl. No.: 16/090,318

(22) PCT Filed: Mar. 23, 2017
(86) PCT No.: PCT/GB2017/050827

§ 371 Date: Oct. 1, 2018

(30) Foreign Application Priority Data
Apr. 1, 2016 (GB) 1605622.8

(51) Int. Cl. A61B 90/00; A61M 29/02

(52) U.S. Cl. CPC A61B 90/02; A61B 17/02; A61M 29/02

(57) ABSTRACT

The present invention provides a tissue expander (10) comprising: a self-inflating core (12), having a non-inflated state and an inflated state, a top surface (14), a bottom surface (16) and a side surface (18); a circumference (C) and having a longitudinal axis X and a coating (20) covering said surfaces (14, 16, 18) and surrounding said core (12), in which said coating (20) comprises a top portion (22), a bottom portion (24) and a side portion (26); wherein, said side portion (26) comprises a concertinaed structure comprising one or more axially spaced circumferentially extending interconnected rings (28) each extending around said axis X and each being spaced from each other along said axis X.

The present invention relates to expandable components and relates particularly but not exclusively to components known as tissue expanders. Such expanders are used by medical, veterinary and dental staff to cause the expansion of patient tissue in advance of corrective or supplemental surgery and generally are such as to have a controlled rate of expansion and may also be configured to have a delayed expansion capability. In one aspect the present invention relates particularly to the inter-relationship of an outer coating over an inner expandable material in which the outer coating is provided with non-elastic expansion capabilities such as to allow the inner expandable material to expand whilst limiting tension in the outer coating.

One known form of tissue expander is described in EP2470233 and from which it will be appreciated that they generally comprise a dried then compressed material which, when re-hydrated will expand to re-gain any height lost during compression. The rate of expansion may be controlled by coating the expandable material in a bio-degradable coating which, upon insertion in the patient, will degrade upon exposure to moisture and then allow the moisture to access the expandable de-hydrated material such as to hydrate it and cause it to expand. An alternative approach is to make the expandable material as an Interpenetrating Network of bio-degradable material and expandable material which, in combination, provide the delay and the expansion. The expandable material can be formed from a number of materials but new forms of hydrogel (a gel in which water is the dispersion medium) are of particular use in such applications. Such expanders are generally known as “self-inflating tissue expanders”.

Whilst the above-mentioned arrangement is able to offer some degree of control over the delay and the rate of expansion, it has been found that still further control is required in order to provide an acceptable product. One possible solution is available in GB1314707 which provides a tissue expander comprising a self-inflating core having a non-inflated state and an inflated state and a water impermeable coating surrounding said core, in which the coating includes a plurality of first and/or second apertures through said coating which, in operation, act to control the rate of water ingress and allow for the control of any expansion by altering the size, number and possibly also the position of the holes themselves. The invention described in GB1314707 relates most particularly to the particular properties of the expandable material itself in combination with the provision of the holes through the water impermeable layer.

WO 2015/022536 discloses a tissue expander having an inner core of self-inflating material and an outer coating of fluid impermeable material which, upon expansion of the inner core stretches to accommodate the expansion.

WO 2005/030092 discloses a self-expanding stent having a plurality of self-expanding rings but fails to disclose an expandable inner core.

WO 01/52774 discloses a prosthetic breast form having a non-expandable inner core and an outer casing which is not concertinaed.

US 2008/306505 discloses a soft tissue expander with a concertinaed structure but without a self-expanding core.

WO 95/26696 discloses a soft tissue expander with a concertinaed structure but without a self-expanding core.

Whilst the above product provides a certain level of improvement, it has been found that a further improvement can be obtained by controlling the inter-relationship of the outer coating over the inner core of expandable material by providing the outer coating with non-elastic expansion capabilities such as to allow the inner expandable material to expand whilst limiting tension in the outer coating.

Accordingly, the present invention provides A tissue expander comprising: a self-inflating core having a non-inflated state and an inflated state, a top surface, a bottom surface and a side surface; a circumference and having a longitudinal axis X and a coating covering said surfaces and surrounding said core, in which said coating comprises a top portion, a bottom portion and a side portion; wherein, said side portion comprises a concertinaed structure comprising one or more axially spaced circumferentially extending interconnected rings each extending around said axis X and each being spaced from each other along said axis X wherein one or more of said one or more rings may comprise an edge having a thickness T1, and a pair of side portions of thickness T2, said side portions having a first end connected to said edge and a second end remote therefrom and wherein the thickness T2 of each side portion is less than thickness T1 of edge.

In one arrangement, the thickness T2 of each side portion is substantially half the thickness T1 of the edge.

In one arrangement, said edge is radially outward of said side portions and said side portions extend radially inwardly of said edge. In an alternative arrangements said edge is radially inward of said side portions and said side portions extend radially outwardly of said edge.

The one or more of said side portions may diverge at an angle α from a perpendicular P to said longitudinal axis X. Alternatively, both of said side portions may diverge at an angle α from a perpendicular P to said longitudinal axis X.

The expander may include a plurality of adjacent axially spaced circumferentially extending interconnected rings and wherein each of said rings comprise an edge, and a pair of side portions, said side portions having a first end connected to said edge and a second end remote therefrom and wherein said second ends are each connected to corresponding second ends of an adjacent ring.

The top and bottom portions of said coating may each connected to second ends of said one or more ring.

The expander may comprising a single ring having a spiral form which spirals around said longitudinal axis X and extends from the top portion to the bottom portion of the coating. The coating may comprises a flexible material. The coating may also comprise a stretchable material. The coating may comprise a fluid impermeable material having one or more apertures therein. The coating may comprise a moulded coating moulded around the core.

The inner core may comprise a water permeable hydrophilic material.

The expander may include a semi-permeable barrier material within one or more apertures or controlling the rate of water ingress.

The expander may exist and be sold in a non-expanded state, a partially expanded state or in a fully expanded state.

The core may comprises a hydrophilic self-inflating polymer.

The angle α may be between 35 degrees and 65 degrees.

The present invention will now be more particularly described by way of example only with reference to the accompanying drawings, in which:

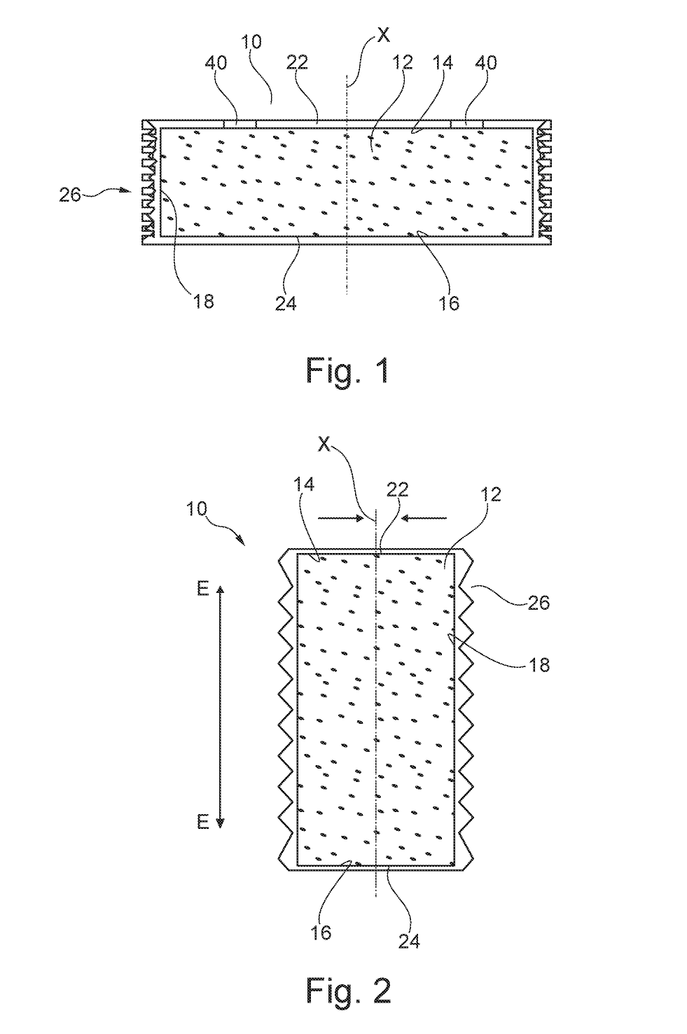

FIG. 1 is a cross-sectional view of a first embodiment of the present invention shown in a compacted and pre-expanded state;

FIG. 2 is a cross-sectional view of the embodiment of FIG. 1 shown in an expanded state;

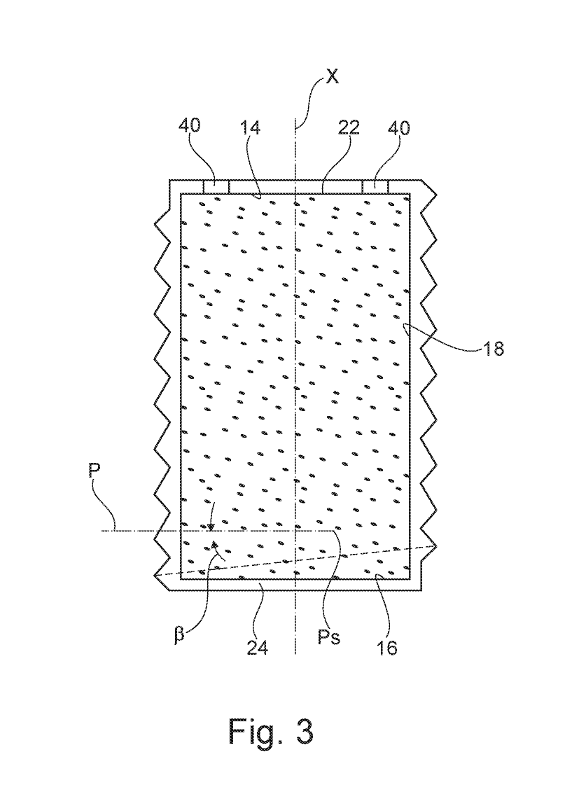

FIG. 3 is a cross-sectional view of a second embodiment of the present invention which differs from the first solely by the provision of a spiral side portion;

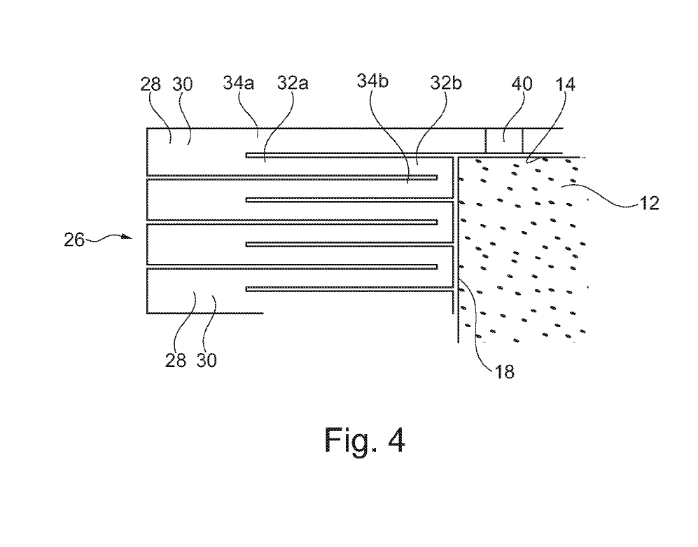

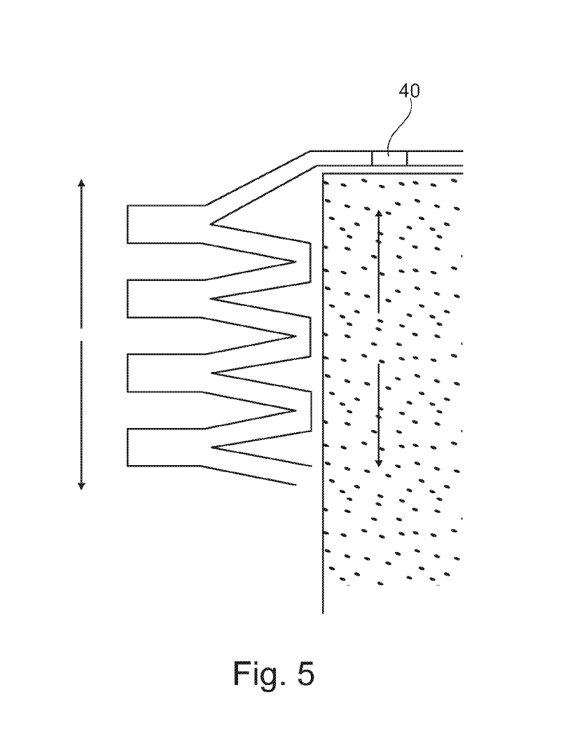

FIG. 4 is a close-up cross-sectional view of a top portion of the side-portion of the first and second embodiments of FIGS. 1 to 3 showing the side portion in a compacted state;

FIG. 5 is a close-up cross-sectional view of the top portion shown in FIGS. 1 to 3 when in a partially expanded state;

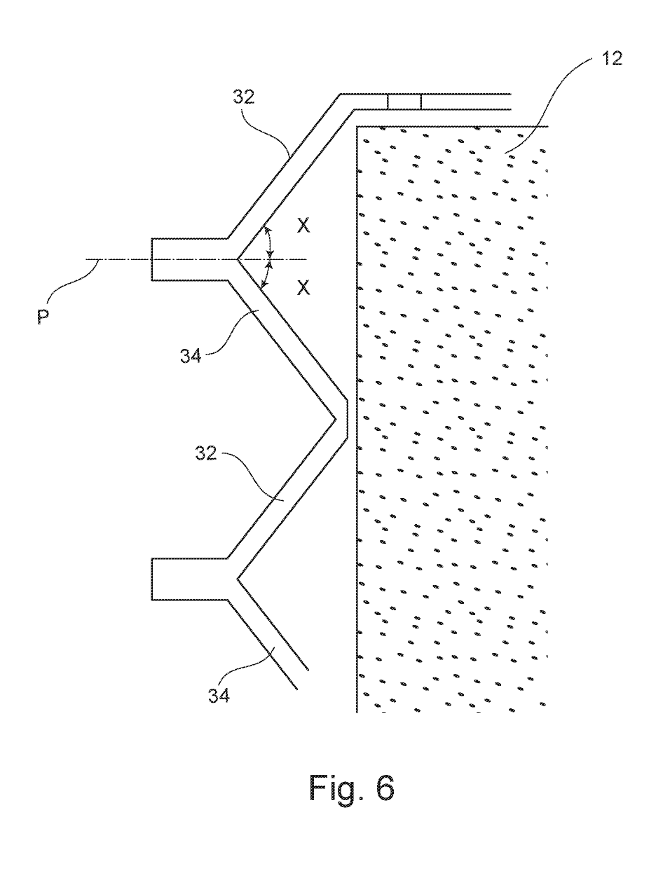

FIG. 6 is a close-up cross-sectional view of the top portion of FIGS. 1 to 3 when in a fully expanded state; and

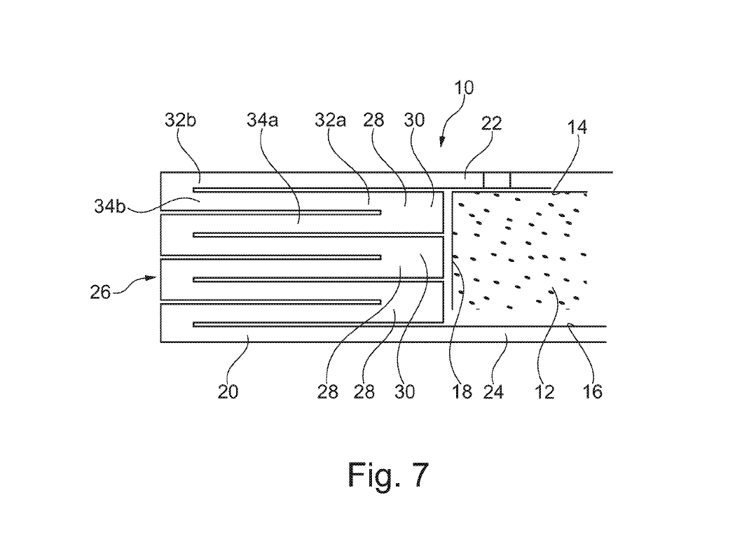

FIG. 7 is a close-up cross-sectional view, the reverse of FIG. 4.

Referring now to the drawings in general but particularly to FIGS. 1 and 2, a tissue expander (10) comprises an expandable core (12) of self-inflating material discussed in detail later herein which includes a top surface (14), a bottom surface (16) and a side surface (18) having a circumference C and having a longitudinal axis X. A coating (20) covers the surfaces (14), (16) and (18) and surrounds the core (12) and comprises a top portion (22), a bottom portion (24) and a side portion (26) which, between them cover each of the top surface (14), bottom surface (16) and side surface (18) of the core (12). The coating (20) is continuous and acts as a barrier between any environment surrounding the tissue expander (10) and the core (12), the function of which will be explained in detail later herein. The side portion (26) comprises a concertinaed structure comprising one or more axially spaced circumferentially extending interconnecting rings (28) each of which extends around axis X and each of which is spaced from each other along said axis X. The concertinaed arrangement of the side portion has the advantage of allowing for greater expansion than a conventional outer casing side portion. Preferably the spacing is even but it will be appreciated that un-even spacing of the rings along axis X may be possible and appropriate. In one arrangement of the invention, the one or more rings (28) comprise or include an edge (30) which has a thickness T1 and further comprises a pair of side portions (32), (34) of thickness T2 which have first ends (32a), (34a) connected to said edge (30) and a second end (32b), (34b) remote therefrom. The thickness T2 is, preferably, less than thickness T1 and the relative thicknesses may be such as to impart a generally resilient form to the ring (28) and a generally flexible form to the side portions (32), (34) such as to allow the side portions to flex and bend relative to the ring (28). In a particular arrangement the thickness T2 of each side portion (32), (34) is substantially half the thickness T1 of the edge. Such an arrangement will allow the side portions (32), (34) to fold in on top of each other and, in a compacted arrangement, nestle within the confines of the thickness T1, as best seen in FIG. 4.

It will be appreciated that the edge (30) may be radially outward of the side portions (32), (34) or radially inwards thereof. The former arrangement is shown in FIGS. 4 to 6 whilst the latter arrangement is shown in FIG. 7 which is the reverse equivalent of the arrangement of FIG. 4. The former arrangement has the advantage of the resilient rings protecting the more delicate side portions (32), (34) whilst the latter arrangement has the advantage of providing a softer exterior arrangement.

As shown generally in the drawings, the arrangement of the side portions (32), (34) is, preferably, such as to have the side portions (32), (34) diverge at an angle α from a perpendicular P to said longitudinal axis when said core (12) is in a partially or completely expanded state, as best seen in FIGS. 2, 5 and 6. Such an arrangement will reduce tension on the side portions (32), (34) when under loads created by the expansion of the core (12) whilst the relative flexibility of the side portions (32), (34) will allow the side portions (32), (34) to be compressed into the relatively flat arrangement of FIG. 4 but without exerting excessive loading on the joint formed at the first end (32a), (34a) with the edge (30). In effect, the side portions will move from a slightly compressed arrangement as shown in FIG. 4 to an intermediate position best seen in FIG. 5 in which the side portions (32), (34) are either under no load or relatively unloaded and then to a slightly expanded state best seen in FIG. 6 in which the side portions (32), (34) may be under some tension. Alternatively, the side portions and angle α may be such that the side portions (32), (34) are under no tension or compression when the core (12) is in the fully expanded state of FIG. 6 but under slight compression when in the partially expanded state of FIG. 5 and under slightly greater compression when in the non-expanded state of FIGS. 1 and 4. It will be appreciated that one or other or both of side portions (32), (34) may diverge at angle α to the perpendicular P.

Whilst the expander (10) may comprise just a single ring (28), it has been found that providing a plurality thereof is particularly advantageous. Such an arrangement comprises a plurality of adjacent axially spaced (along axis X) circumferentially extending interconnecting rings (28) wherein each of the rings (28) comprise an edge (30) and a pair of side portions (32), (34) as discussed in detail above. Each of said side portions (32), (34) have a first end (32a, 34a) connected to said edge (30) and a second end (32b, 23b) remote therefrom and said second ends (32b, 34b) are each connected to corresponding second ends (32b, 34b) of an adjacent ring (28) such as to form a multiple ring concertinaed structure able to accommodate greater expansion than a single ring concertinaed structure as discussed earlier.

In either of the arrangements described above, the top and bottom portions (22, 24) of the coating (20) are each connected to second ends (32b, 34b) of said one or more side portions (32, 34).

In an alternative arrangement, the concertinaed structure may comprise a spiral form as best seen in FIG. 3 which, in effect, has a single ring (28) having a spiral form which spirals around said longitudinal axis X and extends from the top portion (22) to the bottom portion (24) of the coating (20). It will be appreciated that the cross-sectional arrangement of the ring (28) in the spiral arrangement of FIG. 3 will be the same as that of the arrangements of FIGS. 1, 2 and 4 to 7 save for the fact that the perpendicular axis P will be replaced by an axis of the spiral P_{S} which lies at an angle β relative to the perpendicular P.

Whilst the coating (20) may comprise any one of a number of flexible materials such as polymeric materials including silicon, it has been found that there is some advantage in providing the coating (20) in the form of a flexible and stretchable material such as to allow for the flexing of the coating (20) during insertion of the expander (10) into a patient and possibly also any excess expansion of the core (12) should that occur. In the preferred arrangement the coating (20) comprises a fluid impermeable coating and may comprise a moulded coating moulded around the core (20).

The expansion of the core (12) is dependent upon the core (12) being exposed to moisture and, consequently, it is desirable to add one or more holes or apertures (40) through the coating (20) such as to allow for the passage of fluid to the core (12) as and when desired after insertion. Such an arrangement is well known from the applicants own earlier patents (e.g. EP2470233) and, therefore, not described in detail herein save to say that such apertures may be left open or may be filled by a semi-permeable barrier material (44) for controlling the rate of fluid passage and, hence, the expansion of the core. Whilst there are several materials which poses the self-inflating functional requirements of the core (12), it has been found that hydrophilic self-inflating polymers are very suitable to most applications that the expander may be used for. Consequently, such materials form the preferred option in relation to the present invention.

It will be appreciated that the expander (12) as described above may be sold in the non-expanded state, a partially expanded state or a fully expanded state and the present application and claims protects each state. It will also be appreciated that one may vary the angle α may be selected so as to optimise performance of the product and may easily vary between zero degrees and 65 degrees, preferably between 35 degrees and 65 degrees and most preferably between 40 degrees and 50 degrees.

In operation, the above-mentioned expander (10) will be placed under the skin of an animal or human patient (not shown) through a small incision made by the surgeon. In its non-expanded state, the expander (10) offers little if any resistance to insertion and, hence, can be inserted through a relatively small incision. Once inserted, the expander (10) will be subjected to any moisture present in the immediate vicinity and such moisture will transfer in a controlled manner through apertures (40) and, where provided, any semi-permeable barrier material (44) and thence into the core (12). Upon being exposed to moisture, the core (12) will commence its expansion in the manner well known in the art and, therefore, not described herein, but such expansion is relatively slow and controlled. The degree of control over expansion can be modified by amending the number, size and location of any apertures (40) and amending the presence or absence or material properties of any semi-permeable barrier material (44). As the expansion progresses, the expander (10) will cause the side portions (32, 34) of the ring or rings (28) to open out and, hence, expansion is accommodated in the direction of arrows E-E of FIG. 2. FIGS. 4 to 6 should be referred to for a visual representation of the expansion from the fully compacted state of FIG. 4 to the fully expanded state of FIG. 6.

1. A tissue expander comprising:
a) a self-inflating core, having a non-inflated state and an inflated state, a top surface, a bottom surface and a side surface; a circumference (C) and having a longitudinal axis X and
b) a coating covering said surfaces and surrounding said core, in which said coating comprises a top portion, a bottom portion and a side portion;
wherein, said side portion comprises a concertinaed structure comprising one or more axially spaced circumferentially extending interconnected rings each extending around said axis X and each being spaced from each other along said axis X, and wherein one or more of said one or more rings comprise an edge having a thickness T1, and a pair of side portions of thickness T2, said side portions having a first end connected to said edge and a second end remote therefrom and wherein the thickness T2 of each side portion is less than thickness T1 of edge.

2. A tissue expander as claimed in claim 1, wherein the thickness T2 of each side portion is (substantially) half the thickness T1 of the edge.

3. A tissue expander as claimed in claim 1, wherein said edge is radially outward of said side portions and wherein said side portions extend radially inwardly of said edge.

4. A tissue expander as claimed in claim 1, wherein said edge is radially inward of said side portions and wherein said side portions extend radially outwardly of said edge.

5. A tissue expander as claimed in claim 3, wherein one or more of said side portions diverge at an angle α from a perpendicular P to said longitudinal axis X.

6. A tissue expander as claimed in claim 3, wherein both of said side portions diverge at an angle α from a perpendicular P to said longitudinal axis X.

7. A tissue expander as claimed in claim 1, and comprising a plurality of adjacent axially spaced circumferentially extending interconnected rings and wherein each of said rings comprise an edge, and a pair of side portions, said side portions having a first end connected to said edge and a second end remote therefrom and wherein said second ends are each connected to corresponding second ends of an adjacent ring.

8. A tissue expander as claimed in claim 7, wherein said top and bottom portions of said coating are each connected to second ends of said one or more rings.

9. A tissue expander as claimed in claim 1, and comprising a single ring having a spiral form which spirals around said longitudinal axis X and extends from the top portion to the bottom portion of the coating.

10. A tissue expander as claimed in claim 1, wherein said coating comprises a flexible material.

11. A tissue expander as claimed in claim 1, wherein said coating comprises a stretchable material.

12. A tissue expander as claimed in claim 1, wherein said coating comprises a fluid impermeable material having one or more apertures therein.

13. A tissue expander as claimed in claim 1, wherein said coating comprises a moulded coating moulded around the core.

14. A tissue expander as claimed in claim 1, wherein said inner core comprises a water permeable hydrophilic material.

15. A tissue expander as claimed in claim 1, and including a semi-permeable barrier material within one or more of said apertures (for controlling the rate of water ingress).

16. A tissue expander as claimed in claim 1 when in a non-expanded state.

17. A tissue expander as claimed in claim 1 when in a partially expanded state.

18. A tissue expander as claimed in claim 1 when in a fully expanded state.

19. A tissue expander as claimed in claim 1, wherein said core comprises a hydrophilic self-inflating polymer.

20. A tissue expander as claimed in claim 5, wherein said angle α is between 35 degrees and 65 degrees.

21. A tissue expander as claimed in claim 4, wherein one or more of said side portions diverge at an angle α from a perpendicular P to said longitudinal axis X.

22. A tissue expander as claimed in claim 4, wherein both of said side portions diverge at an angle α from a perpendicular P to said longitudinal axis X.

23. A tissue expander as claimed in claim 6, wherein said angle α is between 35 degrees and 65 degrees.

24. A tissue expander as claimed in claim 21, wherein said angle α is between 35 degrees and 65 degrees.

25. A tissue expander as claimed in claim 22, wherein said angle α is between 35 degrees and 65 degrees.
